(19) United States
(12) Patent Application Publication
(10) Pub. No.: US 2020/0390551 A1
(43) Pub. Date: Dec. 17, 2020

(54) Transcatheter Annuloplasty System And Method

(71) Applicant: NORTHWESTERN UNIVERSITY, Evaston, IL (US)

(72) Inventor: Patrick M. McCarthy, Winnetka, IL (US)

(73) Assignee: NORTHWESTERN UNIVERSITY, Evaston, IL (US)

(21) Appl. No.: 16/900,781

(22) Filed: Jun. 12, 2020

Related U.S. Application Data
(60) Provisional application No. 62/860,646, filed on Jun. 12, 2019.

(51) Int. Cl. A61F 2/24

(52) U.S. Cl. CPC A61F 2/2445; A61F 2/2466; A61F 2230/0091; A61F 2250/0007; A61F 2220/0016; A61F 2210/0023

(57) ABSTRACT

An annuloplasty device that includes at least one helical coil usable for percutaneous implantation and activatable such that a length of the coil shortens, thereby reducing a circumference of a dilated valve annulus.

### RELATED APPLICATIONS

This application claims priority to U.S. Provisional Application Ser. No. 62/860,646 filed Jun. 12, 2019 entitled Transcatheter Annuloplasty System And Method, which is hereby incorporated herein by reference in its entirety.

### FIELD OF THE INVENTION

The present invention pertains to a transcatheter annuloplasty ring anchoring system therefore.

### BACKGROUND OF THE INVENTION

Annuloplasty rings and various methods of implanting such rings for the treatment of heart valve disorders has been known for several years, and represents a significant advancement in the treatment of heart disease. Annuloplasty rings are sutured, clipped, or otherwise secured to the patients valve annulus and are used to reduce the diameter of an enlarged or diseased heart valve, thereby allowing the valve leaflets to establish or reestablish coaptation, thereby reducing or eliminating regurgitant flow through the valve.

Historically annuloplasty rings have required open heart surgery to install. Surgery provides the surgeon access to suture the ring in place within the valve annulus. However, surgery has inherent risks. Recovery from open heart surgery can also be extensive.

The very nature of an annuloplasty ring has made the successful development of a percutaneously-implanted ring difficult. The ring must be affixed to the valve annulus in such a manner as to allow the ring to pull the annulus into a configuration having a smaller diameter. This force requires secure attachment around the entire perimeter of the ring and all of the forces placed on the annulus are done so by the anchors. This is a much more complicated problem than typical percutaneously-delivered implants, such as stents, which expand and place a force on the native tissue that results in a self-anchoring configuration.

As a result, there is a distinct need for an annuloplasty ring that includes a novel anchoring system that does not require open-heart surgery while providing a pulling force on the valve annulus without risk of dislodgement or tissue damage.

### SUMMARY OF THE INVENTION

Described herein are annuloplasty rings for transcatheter implantation. The rings provide novel anchoring mechanisms that follow that engage the valve annulus and allow for an atraumatic, yet effective pulling force. The anchor embodiments also provide inherent spring-like characteristics to facilitate “valve in ring,” implantation, which is the subsequent implantation of a valve into the existing ring.

One aspect of the invention provides a transcatheter implantation that includes anchors that surround a ring.

Another aspect is an anchoring spring that can be activated. The anchors are spiral shaped and can be rotated into the tissue around the ring because the leading edge is tapered and sharp like the needle on a surgical suture. After the anchor is placed, it can be activated to contract, shorten, and tighten around the ring. Shortening the overall length of the anchor results in a reduced circumference of the dilated annulus. The anchor thus secures the ring and decreases annular dilation. In at least one embodiment, the anchor is activated by heating the anchor. Heating, in at least one embodiment, can be accomplished by the recipient's body temperature.

In one aspect, multiple anchors may be used. In at least some of these embodiments, the first anchor may be contiguous with a leading edge of the ring, such that the first anchor is rotated into the tissue. Subsequent anchors are then advanced down the outer lumen of a double lumen delivery system, rotated, activated, and secured around the ring.

Another aspect provides a double lumen delivery system.

Another aspect is a method for installing a ring which allows implantation through the femoral vein, jugular vein, directly, or through the right atrium or other vascular structure. The anchor used with this method may have several embodiments, including but not limited to: a plurality of metal or other components; anchors of varying thickness; anchors having varying numbers of “turns” (one or many); and anchors that may be potentially activated by a variety of methods.

Another embodiment involves implanting a spring-like anchor that further serves as the ring. In other words, the anchor spring acts as the annuloplasty device. Ideally, this would be a sequence of connected springs that are activated and would shorten the dilated annulus. This embodiment could utilize a single lumen delivery system.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which

FIG. 1 is a perspective view of an embodiment of an annuloplasty device of the invention;

FIG. 2 is a detailed perspective view of a distal end of the embodiment of FIG. 1;

FIG. 3a is an end view of a step in a sequence of an embodiment of a device of the invention being activated;

FIG. 3b is an end view of a step in a sequence of an embodiment of a device of the invention being activated;

FIG. 3c is an end view of a step in a sequence of an embodiment of a device of the invention being activated;

FIG. 4, is a depiction of an embodiment of an annuloplasty ring of the invention installed in a patient;

FIG. 5 is a depiction of an embodiment of an annuloplasty ring of the invention installed in a patient; and,

FIG. 6 is a perspective view of a delivery device for an embodiment of the invention.

### DESCRIPTION OF THE EMBODIMENTS OF THE INVENTION

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements.

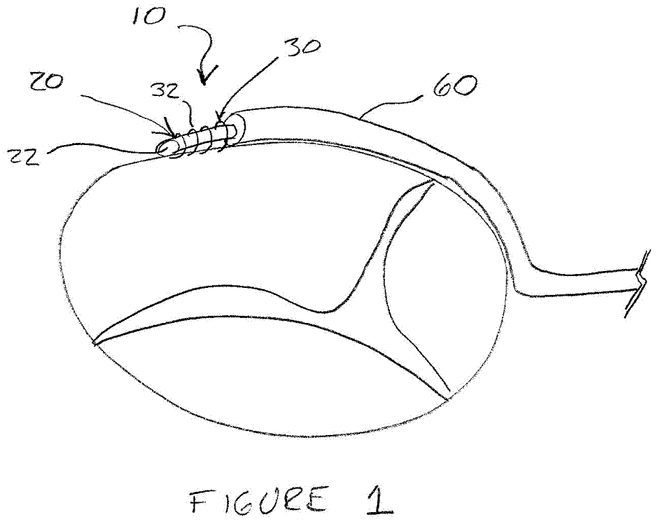

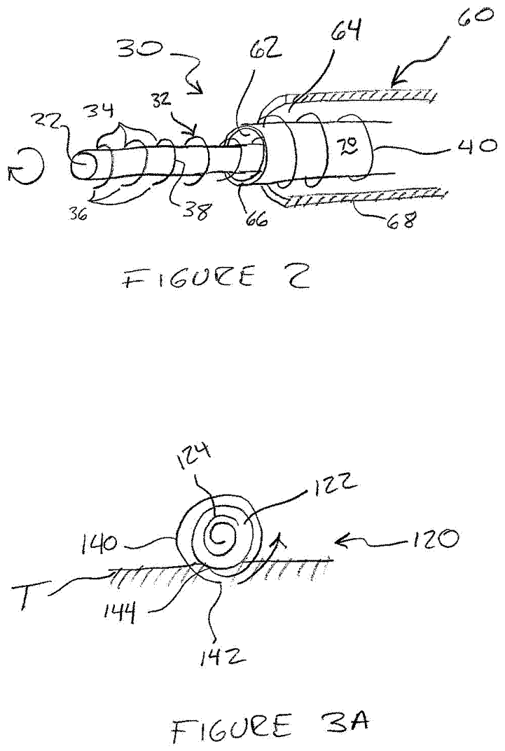

Referring now to the figures, and first to FIGS. 1 and 2, there is shown an embodiment 10 of an annuloplasty ring and delivery system of the invention. The system 10 generally comprises a ring 20, an anchor mechanism 30 and a delivery catheter 60.

The ring 20 generally includes an elongate core 22 that extends between a distal end 24 and a proximal end (not shown in FIG. 1 as the proximal end is still contained within the catheter 60). The core 22 provides a predetermined degree of rigidity and shape to the device and may act upon the anchor mechanism 30 to place the anchor mechanism 30 into a desired shape and size.

In at least one embodiment, the core 22 comprises a memory metal component, such as Nitinol, that allows the core 22 to be straightened and placed in the delivery catheter 60, and to reassume a desired shape once delivered/released from the catheter.

FIGS. 1 and 2 also show an embodiment of an anchor mechanism 30 of the invention. The anchor mechanism 30 includes inner anchor coils 32, and outer anchor coils 40. The inner anchor coils 32 are contiguous, and extend from the core 22. The inner anchor coils 32 have attached ends 34 and sharpened distal ends 36 and circular or helical bodies 38 that extend between the attached ends 34 and the distal ends 36.

The attached ends 34 are attached to the core 22 and may extend from the core 22 at a shallow, almost tangential, angle. Alternatively, they may extend at a sharper angle, or even radially from the core, and include a bend that spaces the body 38 away from the core 22 by a desired amount.

In the embodiment shown in FIGS. 1 and 2, the body 38 of the anchor coil 32 follows a circumference of the core 22 and has a slightly larger inner diameter than an outer diameter of the core 22 such that a small gap forms between the core 22 and the anchor coil 32. Thus, a circular shape or a slightly helical shape would serve to form the desired gap.

The sharpened distal ends 36 allow the anchor coils 32 to embed themselves into the surrounding tissue when rotated around a central axis. This is accomplished by rotating the core 22 in a manner described in more detail below.

The anchor mechanism 30 may further include outer anchor coils 40. The outer anchor coils provide a deeper, more secure, attachment to the valve annulus, and thus supplement the inner anchor coils. However, due to the anchoring force provided by the inner anchor coils 32, the outer anchor coils 40 may be fewer in number and spaced apart further and/or less evenly than the inner anchor coils 32 to accommodate variances in anatomical structure and also to provide additional strength to desired locations of the annulus.

The outer coils 40 may include one or multiple turns. By way of non-limiting example, the outer coils 40 shown in FIG. 1 may include multiple segments 42, each having three turns 44. Thus, each of the segments 42 forms a spiral having a sharpened distal end 46. Rotating a spiral in one direction will result in the sharpened distal end 46 penetrating tissue. As the spiral segment 42 continues to be turned, the spiral will self-advance like a corkscrew. Because the outer anchor coils 40 surround the core 22, they will track around the core 22 and will be prevented from migrating from a desired location on the annulus. In other words, the core 22 prevents the outer coils from burying themselves completely into the tissue.

In embodiments using multiple segments 42, it is envisioned that the individual segments 42 may be advanced sequentially toward the distal end of the delivery device 60, as needed, to secure the ring 20 in place.

The delivery system 60 of the embodiment 20 of FIG. 1 includes a double-lumen catheter, in which an inner lumen 62 contains the core 22 and the inner anchor coils 32. A concentric, outer lumen 64 contains the outer anchor coils 40. In at least one embodiment, the inner lumen 62 is defined by an inner catheter 66 that is independent of an outer catheter 68 that defines the outer lumen 64. The independent inner catheter 66 would thus at least be able to be moved axially within the outer catheter 68. In at least one embodiment, the inner catheter 66 would further be able to be rotated within the outer catheter 68.

In at least one embodiment, the outer anchor coils 40 are removably attached at one end to an outside surface 70 of the inner catheter 66. This embodiment allows the implantation of the outer anchor coils 40 to be accomplished by rotating the inner catheter 66. In other embodiments, the outer anchor coils 40 are friction fit to the inner catheter 66 such that they may be slid axially down the catheter, such as with a pusher catheter, but will rotate when the inner catheter 66 is rotated, thus allowing implantation.

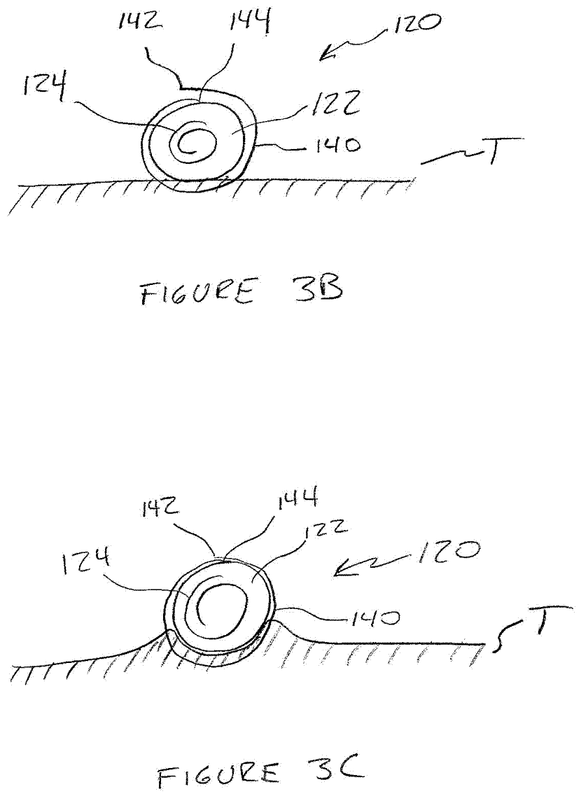

FIGS. 3A-3C show an end view of an embodiment 120 of the invention undergoing an activation process. In FIG. 3A, there is shown an end view of a ring 120 having a core 122 containing an inner coil 124. The ring 120 is being attached to the tissue T of a valve annulus by rotating the ring in the direction shown in the arrow.

The ring has an anchor coil 140 extending around its outside perimeter. The anchor coil has a sharpened tip at one end 142 and is connected to the ring at the other end 144. Due to the helical or spiral shape of the anchor coil 140, rotating the ring in the direction shown in the arrow drives the sharpened end 142 through the tissue T until it reemerges from the tissue.

In FIG. 3B, the ring has been rotated 180 degrees such that the distal sharpened tip 142 of the anchor coil 140 is now located at a top of the ring as oriented in the Figure. Having established a secure anchor to the tissue, the ring may now be activated.

In FIG. 3C, the ring, or more specifically, the outer anchor 140, is activated by heating the anchor coil 140. The anchor coil 140 may be constructed of a memory metal or similar material that responds to heat. The heat may be a powered heat source or the materials may be selected such that activation is caused by body heat.

Activating the anchor coil or coils 140 causes the anchor coil to contract, shorten, and tighten around the ring. Shortening the overall length of the anchor results in a reduced circumference of the dilated annulus. The anchor thus secures the ring and decreases annular dilation.

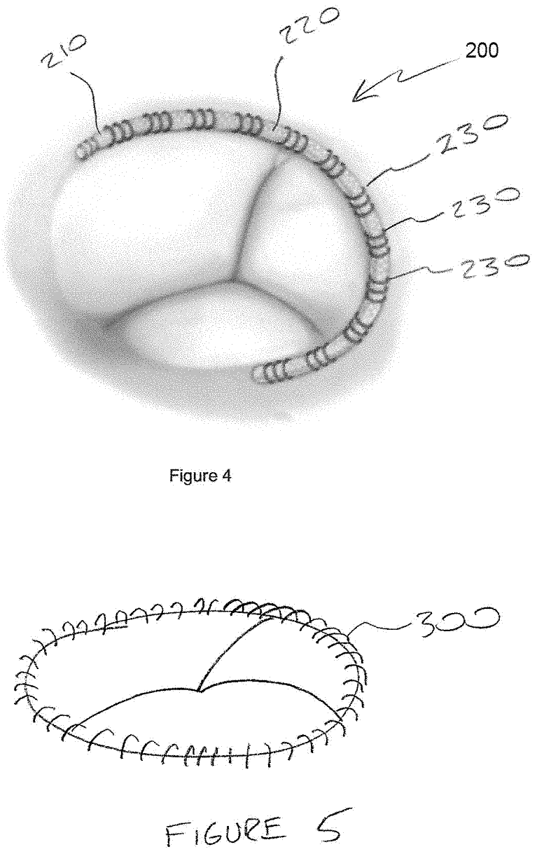

FIG. 4 shows another embodiment of a ring 200. The ring 200 includes a core 210 containing an inner coil 220 and one or more outer anchor segments 230. The inner core 210 is constructed of Nitinol such that after the ring 200 is installed, the inner core 210 may be heated causing the ring 200 to shorten and contract. Like the embodiment of FIGS. 3A-3C, shortening the overall length of the ring results in a reduced circumference of the dilated annulus.

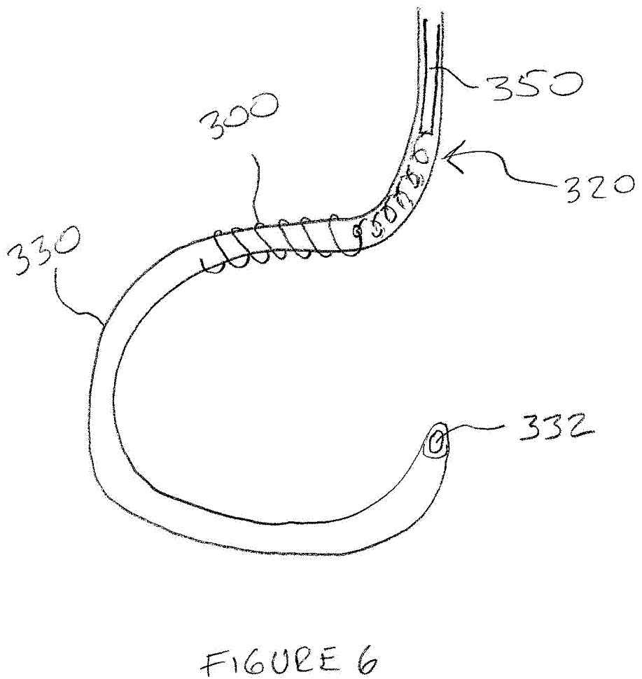

FIG. 5 shows another embodiment of a device 300 of the invention. Rather than a traditional ring, the embodiment 300 comprises a single coil that is implanted around the circumference of the annulus, and then heated, either using power or body heat (i.e. actively or passively) to cause the coil to contract.

FIG. 6 shows a delivery device 320 for the coil 300 of FIG. 5. The delivery device 320 is a shaped catheter 330 with a central lumen 332 containing the coil 300. A side port 340 provides an exit point for a distal end 302 of the coil 300. A pusher catheter 350 is contained within the central lumen 332 and is usable to both advance the coil 300 and also to rotate the coil 300.

The distal end of the catheter 330 is shaped to match the annulus of the target valve. The shape may be memory set into the catheter 330 and later activated by heat. Alternatively, the catheter 330 may be a steerable catheter such that a physician may manipulate the distal end of the catheter into a desired shape under fluoroscopy. If the latter, it would be desirable to be able to lock the shape in place while the coil is being advanced.

Rotating the coil 300 with the pusher catheter 350 causes the distal end 302 of the coil 300 to exit the port 340 and enter the tissue. Further rotation causes the coil 300 to continually engage tissue and to follow the shape of the catheter 330 around the valve annulus as the rotating coil encircles the catheter 330. As such the catheter 330 becomes an internal guide for the coil 300. The coil is rotated until a proximal end of the coil exits the side port 340. The catheter 330 may then be removed from within the implanted coil 300.

In at least some embodiments, the coil 300 is constructed of a Nitinol and can be activated to reduce the circumference of the annulus. Activation may be accomplished passively or actively.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

1. An annuloplasty ring comprising a core and at least one coil surrounding the core and usable to anchor the ring to a valve annulus.

2. The annuloplasty ring of claim 1 wherein at least one of the core and the at least one coil comprises Nitinol.

3. The annuloplasty ring of claim 1 wherein at least one of the core and the at least one coil can be activated to reduce the circumference of the valve annulus.

4. The annuloplasty ring of claim 1 wherein the at least one coil comprises an inner anchor coil and an outer anchor coil.

5. The annuloplasty ring of claim 1 wherein one or more of the at least one coil surrounding the core advances itself through tissue when rotated in engaging proximity thereto.

6. The annuloplasty ring of claim 1 wherein said annuloplasty ring reduces in length when activated.

7. A method of reducing a circumference of a valve annulus comprising:
providing at least one coil;
rotating the at least one coil to cause a sharpened distal end of the coil to engage tissue;
activating the at least one coil to cause the coil to shorten, thereby reducing a circumference of the valve annulus.

8. The method of claim 7 further comprising providing a ring.

9. The method of claim 8 wherein rotating the at least one coil comprises rotating the at least one coil around the ring.

10. The method of claim 7 wherein the at least one coil comprises an at least one outer anchor coil.

11. The method of claim 10 further comprising providing a ring having inner anchor coils extending therefrom.

12. The method of claim 11 further comprising rotating the ring to cause the inner anchor coils to engage tissue prior to rotating the at least one outer anchor coil.

13. The method of claim 7 wherein activating the at least one coil to cause the coil to shorten comprises heating the at least one coil.

14. The method of claim 13 wherein heating the at least one coil comprises actively heating the at least one coil.

15. The method of claim 13 wherein heating the at least one coil comprises passively heating the at least one coil.

16. A system for decreasing a circumference of a valve annulus comprising:
a delivery catheter; and,
at least one helical coil that when rotated, exits the catheter and enters tissue outside of the catheter such that further rotation of the coil results in the coil advancing itself around the valve annulus.

17. The system of claim 16 further comprising a ring around which the at least one helical coil rotates.

18. The system of claim 17 further comprising inner anchor coils extending from the ring for initially attaching the ring to tissue prior to deploying the at least one helical coil.

19. The system of claim 18 wherein said delivery catheter comprises a double-lumen catheter.

20. The system of claim 18 wherein the at least one helical coil shortens when heated.
